(19) United States
(12) Patent Application Publication
(10) Pub. No.: US 2021/0292846 A1
(43) Pub. Date: Sep. 23, 2021

(54) Methods of Assessing the Risk for the Development of a Condition in a Uveal Melanoma (UVM) Patient

(71) Applicant: THOMAS JEFFERSON UNIVERSITY, Philadelphia, PA (US)

(72) Inventors: Isidore Rigoutsos, Astoria, NY (US); Eric Londin, Wynnewood, PA (US); Rogan G. Magee, Philadelphia, PA (US)

(21) Appl. No.: 17/206,796

(22) Filed: Mar. 19, 2021

Related U.S. Application Data
(60) Provisional application No. 62/991,936, filed on Mar. 19, 2020.

(51) Int. Cl. C12Q 1/6886; G16B 25/10

(52) U.S. Cl. CPC C12Q 1/6886; G16B 25/10; C12Q 2600/106; C12Q 2600/178; C12Q 2600/118

(57) ABSTRACT

The present disclosure provides a method for assessing whether a uveal melanoma (UVM) patient has, or is at risk of developing a condition, the method comprising measuring the abundance of at least two isomiRs, and/or at least one tRNA derived fragment (tRF), in a biological sample obtained from the patient, and computing the difference in abundance of the at least two isomiRs and at least one tRF as an indication that the subject either has, or is at risk of developing, or is at a given stage of the condition.

### CROSS-REFERENCE TO RELATED APPLICATIONS

This application claims priority under 35 U.S.C. § 119(e) to U.S. Provisional Patent Application No. 62/991,936, filed Mar. 19, 2020, the contents of which are incorporated by reference herein in their entirety.

### STATEMENT REGARDING FEDERALLY SPONSORED RESEARCH OR DEVELOPMENT

This invention was made with government support under CA195204 awarded by the National Institutes of Health. The government has certain rights in the invention.

### SEQUENCE LISTING

The ASCII text file named “205961-7040US1 Sequence Listing,” created on Mar. 19, 2021, comprising 1,109 Kbytes, is hereby incorporated by reference in its entirety.

### BACKGROUND OF THE INVENTION

Uveal melanoma (UVM) arises from melanocytes of the uveal tract and is the most common primary intraocular malignancy in adults. With approximately 2,500 new U.S. cases annually, UVM accounts for ˜5% of diagnosed melanomas. UVMs occur anywhere within the uveal tract, but the choroid and ciliary bodies are the most frequent locations (˜90%). Approximately 40% patients develop metastases within 10 years, irrespective of treatment type, but this strongly correlates to tumor size and stage of tumor at diagnosis. Currently, the primary treatment for UVM is surgery or radiation. Despite treatment options, uveal melanoma shows a high propensity for liver metastasis, with less than 12 month median survival.

There is a need in the art for practical and reliable tests to diagnose and evaluate uveal melanoma. The present invention addresses this need.

### BRIEF SUMMARY OF THE INVENTION

The present disclosure provides a method for assessing whether a uveal melanoma (UVM) patient has, or is at risk of developing a condition, the method comprising measuring the abundance of at least two isomiRs, and/or at least one tRNA derived fragment (tRF), in a biological sample obtained from the patient, and computing the difference in abundance of the at least two isomiRs and at least one tRF, wherein the difference is an indication that the subject either has, or is at risk of developing, or is at a given stage of the condition.

In certain embodiments, each isomiR of the at least two isomiRs is encoded by a sequence selected from the group consisting of SEQ ID NOs:1-1677. In certain embodiments, each tRF of the at least one tRF is encoded by a sequence selected from the group consisting of SEQ ID NOs:1678-5511.

In certain embodiments, if the abundance of the at least two isomiRs or the abundance of the at least one tRF in the biological sample differ from the abundance of the same molecules in the reference sample, the patient is determined as having, being at risk of developing, or being at a given stage of the condition.

In certain embodiments, the difference comprises a ratio of the abundance of the at least two isomiRs or the at least one tRF in the biological sample and the abundance of the same molecules in the reference sample, wherein the ratio has a log 2 value with an absolute value greater than or equal to 1.

In certain embodiments, the condition is metastasis.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B depict profiling of UVM isomiRs. FIG. 1A: each bar is a miRNA arm and the y-axis is the number of isomiRs produced by that locus. Shown are the top-20 isomiR-producing loci. FIG. 1B: an example of the expression of isomiRs from the miR-140-3p locus. The y-axis shows the abundance for each shown isomiR in reads-per-million (RPM)—note that the axis is logarithmic (base 2). The leftmost box is the ‘0|0’ isomiR. Boxes labeled “green” indicate those isomiRs in which the median expression level is greater than the median expression level of the ‘0|0’ isomiR. Boxes labeled “yellow” represent isomiRs in which the median expression level is less than that of the ‘0|0’ isomiR. The dashed line shows the median expression of the ‘0|0’ isomiR.

FIGS. 2A-2F depict profiling of UVM tRFs. FIG. 2A: percentage of tRFs as a function of genomic origin—mitochondrial (MT) or nuclear (Nuc)—and structural type. Here, the relative distribution of all reads mapping to tRFs is shown as a function of the possible structural types and genomic origins. The tRFs that align to the mature tRNA can belong to one of five structural types and can originate in either the nuclear or mitochondrial genomes. FIG. 2B: relative distribution of all reads mapping to tRFs as a function of the isoacceptors of origin—only the top 10 isoacceptors are shown. FIG. 2C: relative distribution of all reads mapping to tRFs as a function of tRF length, for both nuclear and MT tRFs. Here, only tRFs with lengths between 16 and 30nt across the 80 UVM samples are considered. FIG. 2D: relative distribution of all reads mapping to tRFs as a function of tRF length, for patients that did or did not develop metastasis. FIG. 2E: relative distribution of all reads mapping to tRFs as a function of tRF length, for M3 and D3 patients, respectively. FIG. 2F: relative distribution of all reads mapping to tRFs as a function of tRF length, for patients with or without a mutation in the EIF1AX gene. P-values were determined by t-tests. ** indicates a P-value ≤0.01.

FIGS. 3A-3F depict the differential abundance of various isomiRs with heatmaps showing the most differentially abundant isomiRs for the classification of UVM based upon different clinical attributes (columns within each of FIGS. 3A-3F): patients who have died versus those who were still alive at last clinical visit (column A), patients who developed metastatic disease versus those who do not (column B), M3 or D3 patients (column C), patients with or without a BAP1 mutation (column D), and patients with or without a mutation in either the EIF1AX or SF3B1 genes (column E). Each row corresponds to a different isomiR. For a given column, each cell depicts the log 2 fold change for the respective isomiR. The isomiRs that correspond to the miR-508/514 cluster and the miR-199a/b locus are indicated. White cells indicate that the isomiR was not differentially abundant for that particular comparison. Cells labeled “red” indicate those isomiRs with increased expression in the relevant category whereas cells labeled “green” indicates those isomiRs with decreased expression in the relevant category.

FIG. 3G provides a key and histogram relating color intensity of FIGS. 3A-3F to log 2 fold change and isomiR counts.

FIGS. 4A-4F depict survival analyses of isomiRs and tRFs. Several Kaplan-Meier curves are shown for representative isomiRs and tRFs. In all cases, the samples were grouped based upon whether the expression of the isomiR in question was above the total mean (see labeled lines) or below the total mean (see labeled lines) in that sample. The x-axis is the time to development of metastasis (in days), while the y-axis shows the relative survival function for patients who were segmented in groups based on the abundance of the corresponding molecule. FIGS. 4A-4D show representative isomiRs (SEQ ID NOs: 88, 161, 279, and 27). FIG. 4E and FIG. 4F show representative tRFs (SEQ ID NOs: 1725 and 2921).

FIG. 5 compares the number of isomiRs that are differentially abundant in each of the shown comparisons.

FIG. 6 depicts the differential abundance of different mature miRNA arms in various pairwise comparisons. The heatmap shows miRNA arms with characteristic differential abundance for different clinical attributes (columns): patients with or without a BAP1 mutation, M3 or D3 patients, patients who developed metastatic disease versus those who did not, patients with or without a mutation in either the EIF1AX or SF3B1 genes, and patients who have died versus those who were still alive at last clinical visit. Shown is the log 2 of the fold change for each comparison. Each row represents a different miRNA arm. Cells that are not labeled “red” or “blue” correspond to comparisons where the aggregate isomiR abundance from the respective miRNA arm was not differentially abundant for that particular comparison.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably 10%, and still more preferably +0.1% from the specified value, as such variations are appropriate to perform the disclosed methods.

“Biological sample” or “sample” as used herein means a biological material isolated from an individual. The biological sample may contain any biological material suitable for detecting the desired biomarkers, and may comprise cellular and/or non-cellular material obtained from the individual. A biological sample may be of any biological tissue or fluid. Frequently the sample will be a “clinical sample” which is a sample derived from a patient. Typical clinical samples include, but are not limited to, bodily fluid samples such as synovial fluid, sputum, blood, urine, blood plasma, blood serum, sweat, mucous, saliva, lymph, bronchial aspirates, peritoneal fluid, cerebrospinal fluid, and pleural fluid, and tissues samples such as blood-cells (e.g., white cells), tissue or fine needle biopsy samples and abscesses or cells therefrom. Biological samples may also include sections of tissues, such as frozen sections or formalin fixed sections taken for histological purposes.

The terms “biomarker” or “marker,” as used herein, refers to a molecule that can be detected. Therefore, a biomarker according to the present invention includes, but is not limited to, an oligonucleotide, an oligopeptide, a nucleic acid, a polynucleotide, a polypeptide, a carbohydrate, a lipid, an inorganic molecule, an organic molecule, each of which may vary widely in size and properties. A “biomarker” can be a bodily substance relating to a bodily condition or disease. A “biomarker” can be detected using any means known in the art or by a previously unknown means that only becomes apparent upon consideration of the marker by the skilled artisan.

The term “biomarker (or marker) expression” as used herein, encompasses the transcription, translation, post-transcriptional modification, post-translational modification, and phenotypic manifestation of a gene, including all aspects of the transformation of information encoded in a gene into RNA or protein. By way of non-limiting example, marker expression includes transcription into messenger RNA (mRNA) and translation into protein. Measuring a biomarker also includes reverse transcription of RNA into cDNA (i.e. for reverse transcription-qPCR measurement of RNA levels.).

As used herein, “biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as clinical parameters, as well as traditional laboratory risk factors. As defined by the Food and Drug Administration (FDA), a biomarker is a characteristic (e.g. measurable DNA and/or RNA) that is “objectively measured and evaluated as an indicator of normal biologic processes, pathogenic processes, or pharmacologic responses to a therapeutic intervention or other interventions”. Biomarkers also include any calculated indices created mathematically or combinations of any one or more of the foregoing measurements, including temporal trends and differences.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The terms “abundance” or “level” as used herein means the absolute or relative amount or concentration of one or more biomarkers in a sample as determined by measuring mRNA, cDNA or protein, or any portion thereof such as oligonucleotide, polynucleotide, oligopeptide, or polypeptide.

“IsomiR” as the term is used herein, refers to an isoform of a microRNA (miRNA). IsomiRs are RNA molecules that comprise a small number of nucleotides (e.g. 18 or more nucleotides) and can be produced from the same arm of a given miRNA precursor molecule. Any two isomiRs from the same miRNA arm share many of their nucleotides and differ in their endpoints. An isomiR can be post-transcriptionally modified through the addition of one or more nucleotides.

“Measuring” or “measurement,” or alternatively “detecting” or “detection,” means determining the presence, absence, quantity or amount (which can be an effective amount) of either a given substance within a clinical or subject-derived sample, including the derivation of qualitative or quantitative concentration levels of such substances, or otherwise determining the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “reference level” or “reference abundance” of a biomarker means a level of the biomarker that is indicative of the absence of a particular disease state or phenotype. When the level of a biomarker in a subject is above (or below) the reference level of the biomarker it is indicative of the presence of a particular disease state or phenotype. When the level of a biomarker in a subject is about the same as the reference level of the biomarker it is indicative of a lack of a particular disease state or phenotype.

As used herein, the term “tRNA derived fragment” or “tRF” refers to a fragment a transfer RNA (tRNA) precursor molecule or of a mature tRNA molecule. A tRF is an RNA molecule that comprises a small number of nucleotides (e.g. 16 or more nucleotides) and is a portion of a precursor tRNA or of a mature tRNA. A tRF can be post-transcriptionally modified through the addition of one or more nucleotides.

As used herein, the term “uveal melanoma” or “UVM” refers to a cancer that arises from melanocytes that are resident in the uveal tract. The uveal tract comprises the iris, the choroid, and the stroma of the ciliary body.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

As stated elsewhere herein, Uveal melanoma (UVM) is the most common primary intraocular malignancy in adults, and, in spite of the treatment options available, UVM shows a high propensity for liver metastasis, with a median survival time of less than 12 months. In UVM, molecular disease features strongly correlate with clinical outcomes. For example, chromosomal abnormalities monosomy of chromosome 3 (M3) are commonly detected and associated with increased metastasis and poorer overall prognosis. Loss of function of the 3p21 BRCA Associated Protein 1 (BAP1) gene, either through mutations or decreased expression, correlates with M3 phenotype. Conversely SRSF2/SF3B1 and EIF1AX mutant tumors have distinct copy number alterations and DNA methylation profiles that associate with the better overall prognosis of the disomy 3 (D3) phenotype.

While the genome's contributions to the disease have been well-studied, it is clear that additional unknown disease mechanisms remain. One such mechanism may relate to short non-coding RNAs (ncRNAs). Two categories of short ncRNAs with demonstrated regulatory roles and emerging strong relevance for UVM are the isoforms of microRNAs (miRNAs), and transfer RNA (tRNA) derived fragments.

MiRNAs are the best-studied ncRNAs to date. These ˜22 nucleotide (nts) RNAs modulate the abundance of proteins and of long non-coding RNAs (lncRNAs) in a sequence-dependent manner. As previously shown, one miRNA can target hundreds to thousands of protein-coding genes through target sites in the coding sequence, 5′ untranslated region (UTR), or 3′-UTR of messenger RNAs (mRNAs). MiRNAs operate through several mechanisms, including translational inhibition, disruption of cap-tail interactions, or exonuclease-mediated mRNA degradation.

Early deep-sequencing studies reported expression of multiple isoforms of miRNAs (isomiRs) that were initially dismissed as aberrant. However, it has been shown that isomiRs are produced constitutively and exhibit tissue-specific abundance profiles, and that some miRNA arms produce more than 30 distinct isomiRs. Importantly, the present inventors have demonstrated that isomiRs from the same miRNA target different mRNAs. Thus, isomiR expression greatly increases the number of target mRNAs regulated by miRNAs. Notably, the most abundant isomiR from a given miRNA locus generally varies amongst different tissues. Presumably, this tissue-dependence corresponds to specific regulatory needs.

This last observation becomes even more relevant when considering that the tissue-specific patterns of isomiR expression are modulated further by individual attributes such as a person's sex, race/ethnicity and population origin, as well as disease type and subtype. Several thousand novel tissue-specific and human-specific miRNA loci have been recently described. These novel miRNA loci produce isomiRs that drive tissue-specific regulatory events that are currently-uncharacterized and cannot be captured by mouse models as these sequences do not exist in rodents.

It is now known that tRNAs produce short ncRNAs, named tRNA-derived fragments (tRFs). Studies have shown that tRFs are not products of random degradation. In fact, some tRFs target mRNAs in a miRNA-like manner. Recent work with human tissues have demonstrated that tRFs are produced constitutively and their profiles depend on a person's sex, race/ethnicity, and population origin, as well as on tissue type, tissue state, and disease, in an analogous manner to the isomiR findings. tRFs have also been shown to decoy RNA-binding proteins, and to displace mRNAs from the active ribosome.

While earlier studies looked at miRNAs from the standpoint of locus expression, efforts to investigate the roles of isomiRs and tRFs in UVM are scarce, if at all present.

Thus, the present disclosure provides the findings from an analysis of these two categories of mRNA regulators across the 80 samples of the TCGA UVM cohort. Specifically, the correlation of isomiR/tRF profiles with clinical outcomes was examined. Particular attention was also provided to identify genomic loci that harbor previously unreported miRNA precursors with UVM-specific transcription. IsomiRs, tRFs, and previously-unreported miRNA loci represent novel and uncharacterized regulators of UVM biology.

Without wishing to be limited by theory, it has now been unexpectedly discovered that differences in the abundance of isomiRs and tRFs relative to control may be assessed in order to determine the clinical attributes of uveal melanoma patients. In various aspects and embodiments, the invention provides methods for assessing the state of a uveal melanoma patient, the method comprising measuring the levels of two or more isoforms of one or more miRNAs (isomiRs) and/or tRNA derived fragments (tRFs) in a sample obtained from the patient; determining the differential abundance of the one or more isomiRs and/or tRFs; and comparing the differential abundance of the one or more isomiRs and/or tRFs.

As shown in the figures and the example below, the differential abundance of short non-coding RNAs is diagnostic with respect to a variety of patient attributes as well as prognostic. All of these are contemplated and considered to be within the scope of the various embodiments of the invention. In various embodiments, the clinical attribute may be mortality, cancer metastases, M3/D3 status, BAP1 mutation status, or EIF1AX/SF3B1 mutation status. Assessing the mortality of the patient, as used herein, means assessing the patient's likelihood of survival over a given timeframe, with respect to UVM. M3/D3 status refers to the monosomy (M3) or disomy (D3) of chromosome 3. M3 is associated with increased metastasis and poorer overall prognosis. Similarly, patients with a mutation in the BAP1 gene have an overall higher risk of progressing to metastatic disease.

In various embodiments, the isomiRs or tRFs are compared to an appropriate control. By way of nonlimiting example, when the condition to be assessed is BAP1 mutation status, an appropriate control is the level of the analogous isomiRs or tRFs from a BAP 1 wild-type patient.

The present disclosure provides a method for assessing whether a uveal melanoma (UVM) patient has, or is at risk of developing a condition, the method comprising:
(a) measuring in a biological sample obtained from the patient the abundance of at least one of:
   (i) at least two isoforms (isomiRs) of at least one miRNA; and
   (ii) at least one tRNA derived fragment (tRF);
(b) computing a difference in the abundance of at least one of:
   (i) the at least two isomiRs of at least one miRNA; and
   (ii) the at least one tRF;
in the biological sample as compared to the abundance of the same molecules in a reference sample,
   wherein the difference that results from the computing is an indication that the subject either has, or is at risk of developing, or is at a given stage of the condition.

In certain embodiments, the condition is metastasis.

In certain embodiments, each isomiR of the at least two isomiRs is encoded by a sequence selected from the group consisting of SEQ ID NOs:1-1677.

In certain embodiments, each tRF of the at least one tRF is encoded by a sequence selected from the group consisting of SEQ ID NOs:1678-5511.

In certain embodiments, the abundance of both the at least two isomiRs and the at least one tRF are measured in the biological sample.

In certain embodiments, if the abundance of the at least two isomiRs in the biological sample differ from the abundance of the same molecules in the reference sample, the patient is determined as having, being at risk of developing, or being at a given stage of the condition.

In certain embodiments, the abundance of the at least two isomiRs in the biological sample are greater than the abundance of the same molecules in the reference sample. In certain embodiments, at least one of the at least two isomiRs is encoded by a sequence selected from the group consisting of SEQ ID NO:88 and SEQ ID NO:161.

In certain embodiments, the abundance of the at least two isomiRs in the biological sample are less than the abundance of the same molecules in the reference sample. In certain embodiments, at least one of the at least two isomiRs is encoded by a sequence selected from the group consisting of SEQ ID NO:279 and SEQ ID NO:30.

In certain embodiments, if the abundance of the at least one tRF in the biological sample differ from the abundance of the same molecules in the reference sample, the patient is determined as having, being at risk of developing, or being at a given stage of the condition. In certain embodiments, if the abundance of both of the at least two isomiRs and the the at least one tRF in the biological sample differ from the abundance of the same molecules in the reference sample, the patient is determined as having, being at risk of developing, or being at a given stage of the condition.

In certain embodiments, the difference comprises a ratio of the abundance of the at least two isomiRs in the biological sample and the abundance of the same molecules in the reference sample, wherein the ratio has a log 2 value with an absolute value greater than or equal to 1. In certain embodiments, the difference comprises a ratio of the abundance of the at least one tRF in the biological sample and the abundance of the same molecules in the reference sample, wherein the ratio has a log 2 value with an absolute value greater than or equal to 1.

In certain embodiments, the ratio has a log 2 with an absolute value greater than or equal to 2. In certain embodiments, the ratio has a log 2 with an absolute value greater than or equal to 3. In certain embodiments, the ratio has a log 2 with an absolute value greater than or equal to 4. In certain embodiments, the ratio has a log 2 with an absolute value greater than or equal to 5. In certain embodiments, the ratio has a log 2 with an absolute value greater than or equal to 6. In certain embodiments, the ratio has a log 2 with an absolute value greater than or equal to 7. In certain embodiments, the ratio has a log 2 with an absolute value greater than or equal to 8. In certain embodiments, the ratio has a log 2 with an absolute value greater than or equal to 9. In certain embodiments, the ratio has a log 2 with an absolute value greater than or equal to 10.

In certain embodiments, the measuring comprises subjecting the biological sample to deep sequencing. In certain embodiments, the measuring comprises subjecting the biological sample to a polymerase chain reaction (PCR). In certain embodiments, the PCR can ensure the identity of the endpoints of the molecule being measured. In certain embodiments, the PCR comprises a modified quantitative reverse transcription PCR (qRT-PCR), including but not limited to “dumbbell PCR”. In various aspects and embodiments, the step of determining the differential abundance of the one or more isomiRs and/or tRFs is done by the use of standard tools such as the Significance Analysis of Microarrays for RNA-seq data (SAMseq), the differential expression analysis for sequence count data (DEseq) and others.

In certain embodiments, the method further comprises recommending a therapeutic regimen.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods

### Datasets

Short RNA-seq data and associated clinical information for 80 primary UVM samples were downloaded from the Genomic Data Commons Data Portal.

### Sequence Read Mapping

Sequenced reads were quality trimmed and adaptors removed using the cutadapt tool. The resulting collection of reads was mapped unambiguously using SHRIMP2. Reads were mapped to the human genome assembly hg19/GRCh37 in order to enable comparisons with the TCGA consortium's report on UVM. No insertions or deletions were allowed during mapping. At most one replacement was permitted during mapping.

Identification of Novel miRNAs

To identify novel miRNAs, each UVM dataset was processed independently with miRDeep2, using default parameter settings and a score threshold of ≥1. For all novel miRNA precursors, the most abundant isomiR was identified and labeled the ‘archetype’ sequence for the corresponding locus.

Identification of isomiRs

Each archetype's miRNAs genomic locus was flanked by six nts on either side. Mapped sequence reads that were contained wholly within those wider windows were treated as belonging to the miRNA precursor and are considered further. Distinct reads that had identical 5′ and 3′ endpoints within a miRNA arm were combined into a single isomiR. The union of all distinct sequences that were identified at each miRNA arm comprise the collection of isomiRs arising from the arm. The isomiR nomenclature scheme used herein has been described in Rigoutsos et. al (Cell, 2014, 136:215-233), which is incorporated herein by reference in its entirety. For isomiRs produced by novel miRNAs, the definition of ‘archetype’ used is described elsewhere herein. For isomiRs produced by miRNAs that are already in a pubic database such as miRBase, archetype is defined as the isoform that the database lists for the corresponding miRNA. For example, the isomiR whose 5′ terminus begins one position to the right (+1) of the archetype's 5′ terminus and whose 3′ terminus ends two positions to the left (−2) of the archetype's 3′ terminus is labeled “+1|-2”. The archetype isomiR for a miRNA arm is denoted by “0|0.”

Identification of tRFs

The tRF “license plate” notation, as described in Rigoutsos et. al (Bioinformatics, 2016, 32:2481-2489), which is incorporated herein by reference in its entirety, is used herein

Quantification of miRNAs and isomiRs

IsomiR was quantified abundance in terms of reads per million or RPM. Only reads that were quality trimmed and had their adaptors removed, and could be aligned exactly to miRNA arms, were considered when calculating the RPM value. To calculate the normalized abundance of a miRNA arm, normalized abundances of all isomiRs from the arm were added. Statistically significant miRNAs and isomiRs were identified with the Threshold-seq algorithm whose threshold calculation adapts to sequencing-depth (“adaptive thresholding”). This thresholding was applied separately to each dataset. Additionally, it was required that a miRNA or isomiR exceeded the Threshold-seq's threshold in at least 20 (25%) of the 80 TCGA UVM samples before it entered the analysis.

Quantification of tRFs

RPM values for tRFs were determined as with miRNAs/isomiRs. For parity, all MINTmap-identified tRFs were further subjected to the same Threshold-seq threshold that was established for the isomiRs of the corresponding dataset. Additionally, as with the miRNAs/isomiRs, the analyses considered only tRFs that were present in at least 20 of the 80 TCGA UVM samples.

Identification of Differentially Abundant miRNAs, isomiRs, and tRFs

This analysis was performed using 5000 permutations of the Significance Analyses of Microarrays (SAM) algorithm and using a stringent False Discovery Rate (FDR) of 1% and a log 2 fold change threshold of absolute value ≥1 (i.e., ≥2× fold change, either an increase or a decrease).

### Overall Survival Analysis

To determine overall survival associations with specific miRNAs, isomiRs and tRFs, Kaplan-Meier survival analyses were performed. Briefly, a given molecule split patient samples into an “above mean” and “below mean” group, depending on whether the molecule was expressed at above or below the molecule's total mean value across the samples. Samples were then stratified into these two groups and computed the time to development of metastasis in each group. Log-rank tests describe the significance of any emergent differences.

### Example 1: The miRNAs have Characteristic Profiles in UVM

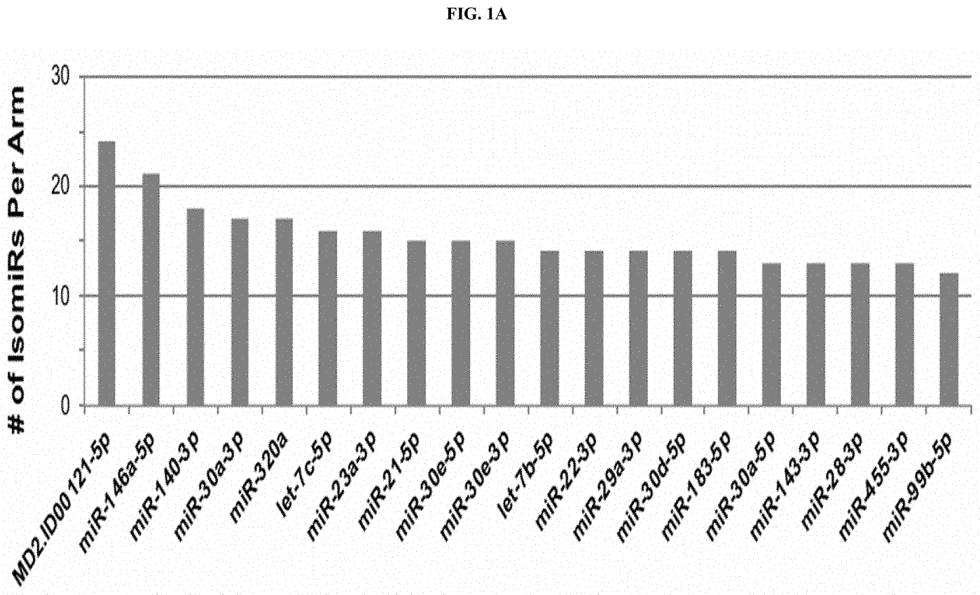

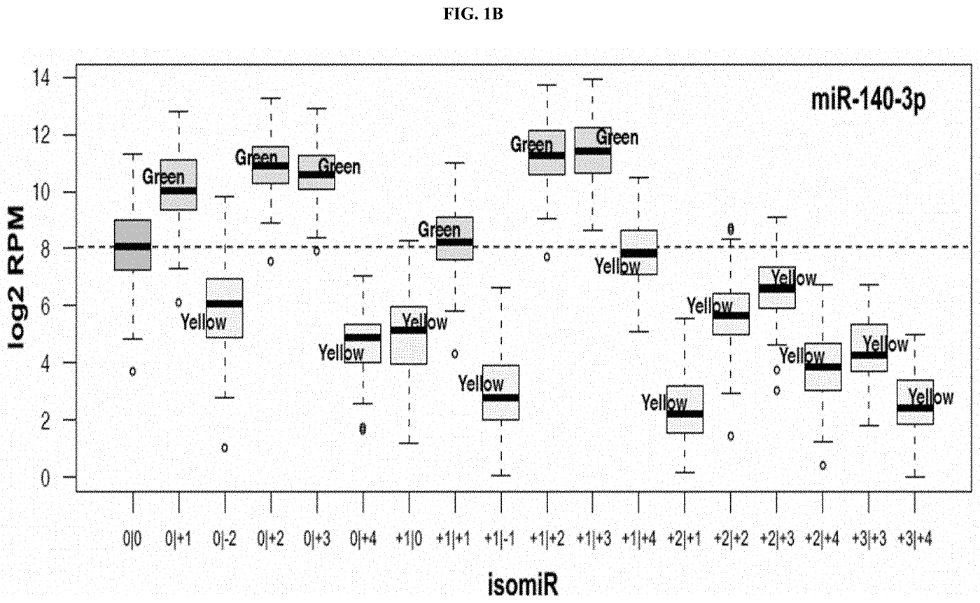

Using stringent criteria described elsewhere herein, isomiRs present in the TCGA UVM cohort were characterized. Four hundred and seven unique miRNA arms, producing one or more isomiRs, are expressed in at least 25% of the 80 samples. Interestingly, 32 appear to be UVM-specific: they have not been reported in any other context. Collectively, 407 arms produce 1,677 statistically-significant isomiRs (Table 1). Of the 407 miRNA arms, 112 produce only one isomiR. Thirty-seven arms produce ≥10 isomiRs. FIG. 1A shows the top 20 isomiR-producing miRNA arms. Notably, some of the best-studied miRNA loci (e.g. miR-21-5p, miR-183-5p, miR-143-3p) produce many isomiRs. Similar to previous findings, increased 3′ isomiR variability in UVM, compared to 5′ endpoints was observed. Notably, the top-producer of UVM isomiRs is MD2.ID00112-5p, a novel miRNA that has been reported previously; MD2.ID00112 currently remains uncharacterized.

TABLE 1
Summary of the UVM miRNAome

| Category | Number |
|---|---|
| miRNA arms previously characterized in UVM | 375 |
| miRNA arms not previously characterized in UVM | 32 |
| Total isomiRs expressed in UVM | 1,677 |
| isomiRs previously linked in UVM (i.e. miRbases 0\|0) | 327 |
| isomiRs not previously linked with UVM | 1,350 |
| isomiRs differing from 0}0 only in their 3′ end | 831 |
| isomiRs differing from 0\|0 only in their 5′ end | 187 |
| isomiRs differing from 0\|0 in both their 5′ end and 3′ ends | 332 |
| miRNA arms with a single isomiR | 112 |
| miRNA arms with ≥ 2 isomiRs | 263 |
| miRNA arms not expressing miRbase (0\|0) isoform | 79 |
| miRNA arms where miRbase isoform is not most abundant | 174 |

For 165 (44%) of the 375 isomiR-producing miRNA arms listed in miRBase, the most abundant isomiR in UVM is not the ‘archetype’. miR-140-3p presents an illustrative example in UVM (FIG. 11n). Four of the five isomiRs that are more abundant than the archetype have “seeds” that differ from the archetype's. Without wishing to be bound by theory, it is because of the different seeds that these four isomiRs may target genes that differ from the genes targeted by miR-140-3p's “0|0” isomiR, thereby contributing to UVM biology in currently uncharacterized ways. Moreover, for another 80 (21.30%) of the 375 miRNA arms in miRBase, the archetype is not present at all in UVMV (Table 1). These observations mirror earlier findings in other tissues and cell types, and lead to an important corollary: focusing on “0|0” isomiRs (miRBase entries) will ignore important regulatory molecules in UVM.

### Example 2: Newly-Discovered miRNA Loci are UVM-Specific

32 new miRNA loci were discovered in the 80 samples (U.S. Provisional Patent Application No. 62/991,936). When compared to the other 31 TCGA cancer types, many of these loci produce isomiRs that are either exclusively present in UVM, or prominently abundant in UVM. These novel sequences are ideal candidates to serve as candidate UVM biomarkers.

### Example 3: tRFs have Characteristic Profiles in UVM

Across the 80 UVM samples, 3,780 unique tRFs have been found. Of these tRFs, 2,286 (60%) are exclusive to the tRNA space, and can only have arisen from tRNA genes. 2,847 (75%) of the tRFs originate in nuclearly-encoded tRNAs, whereas the remaining 933 (25%) derive from mitochondrially-encoded tRNAs. Although the absolute number of tRFs from nuclear tRNAs seems to outweigh that from MT tRNAs, the situation is actually reversed: isodecoder for isodecoder, the 22 MT-encoded tRNAs contribute nine times as many tRFs as the 610 nuclearly-encoded tRNAs.

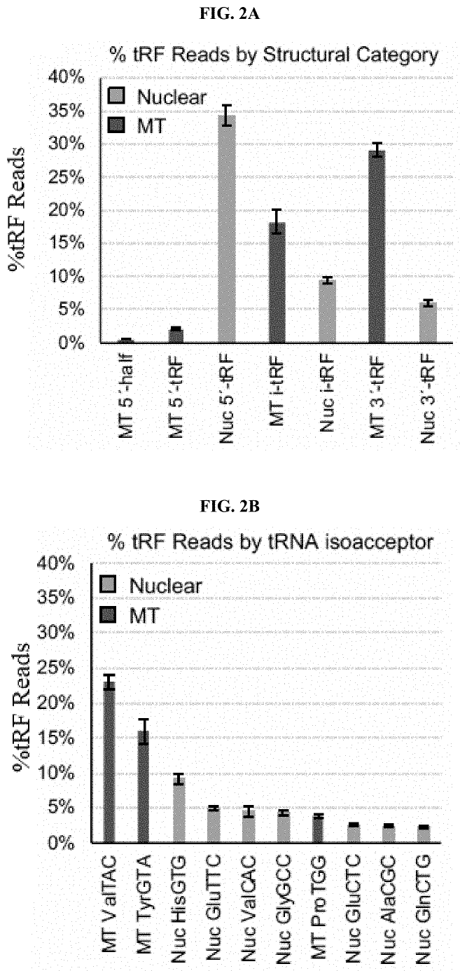

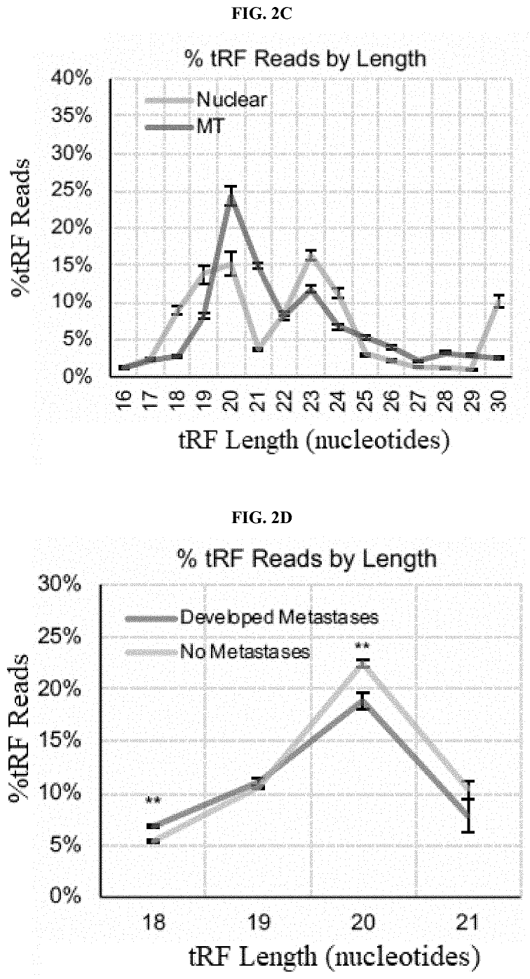

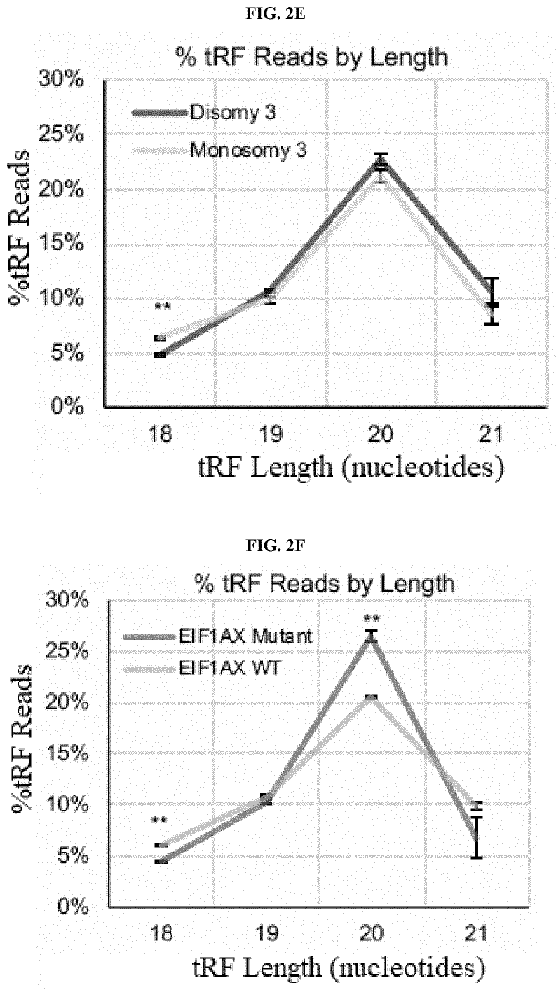

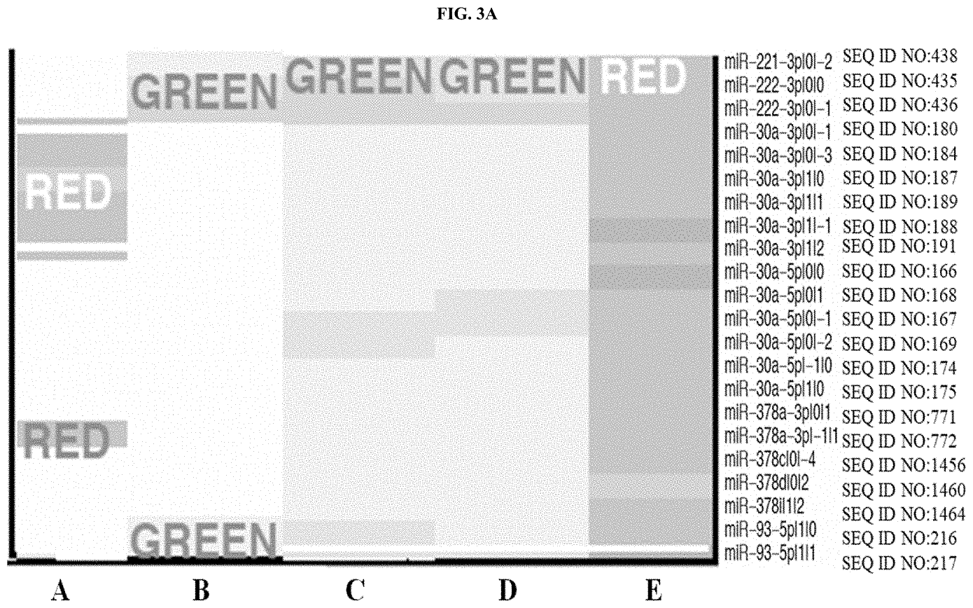

The tRFs that overlap the mature tRNA sequence belong to one of five structural categories: 5′-tRFs, i-tRFs, 3′-tRFs, 5′-tRNA halves (5′-tRHs), and 3′-tRNA halves (3′-tRHs). In the UVM samples, the majority of MT-derived tRFs are either 3′-tRFs or i-tRFs (FIG. 2A). On the other hand, nuclearly-encoded tRNAs give rise predominantly to 5′-tRFs (FIG. 2A). The 5′- and 3′-tRHs are under-represented in the UVM samples: the TCGA sequencing protocol specifically enriched for miRNAs (20-24 nts) and ran for only 30 cycles. Given that tRHs are among the most abundant molecules in cells, it follows that many additional, as yet uncharacterized, tRF species exist within the UVM transcriptome.

Remarkably, 73.15% of the sequenced tRFs are produced by only 10 of the 61 nuclear and 22 MT isoacceptors: MT tRNA^{ValTAC}, MT tRNA^{TyrGTA}, Nuc tRNA^{HisGTG}, Nuc tRNA^{GluTTC}, Nuc tRNA^{ValCAC}, Nuc tRNA^{GlyGCC}, MT tRNA^{ProTGG}, Nuc tRNA^{GluCTC}, Nuc tRNA^{AlaCGC}, and Nuc tRNA^{GlnCTG} (FIG. 2B). This suggests outsized roles for the respective tRNAs. tRFs from MT tRNAs are generally shorter (20-21 nts) whereas tRFs from nuclear tRNAs show peaks at 20 and 23 nts (FIG. 2C). Notably, previous work on the TCGA prostate cancer datasets showed a prominent peak at 18 nts, whereas the triple negative breast cancer TCGA datasets showed the prominent peak at 19 nts. These observations indicate tissue-specific differences in the production of tRFs.

### Example 4: IsomiRs can Serve as Candidate Markers of Disease Progression and Patient Survival

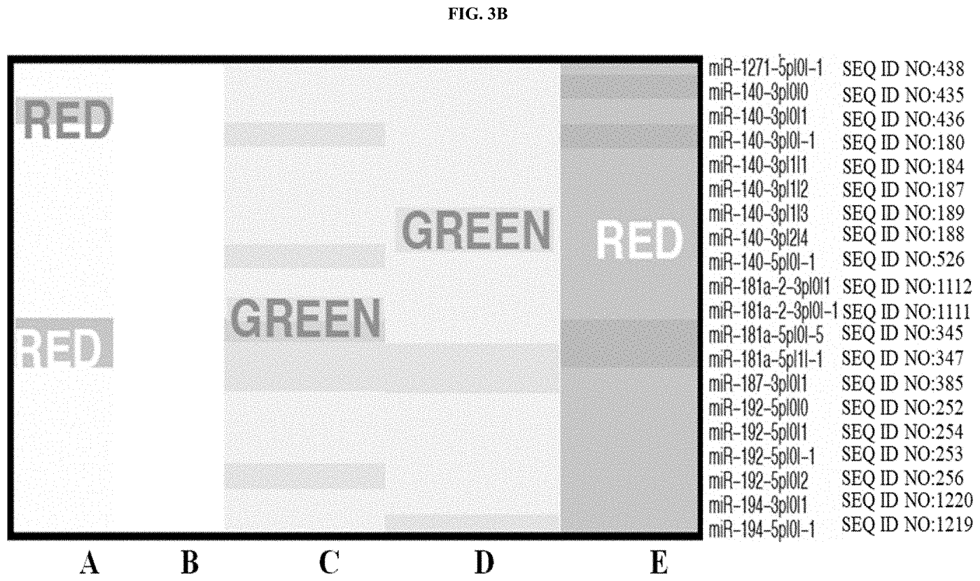

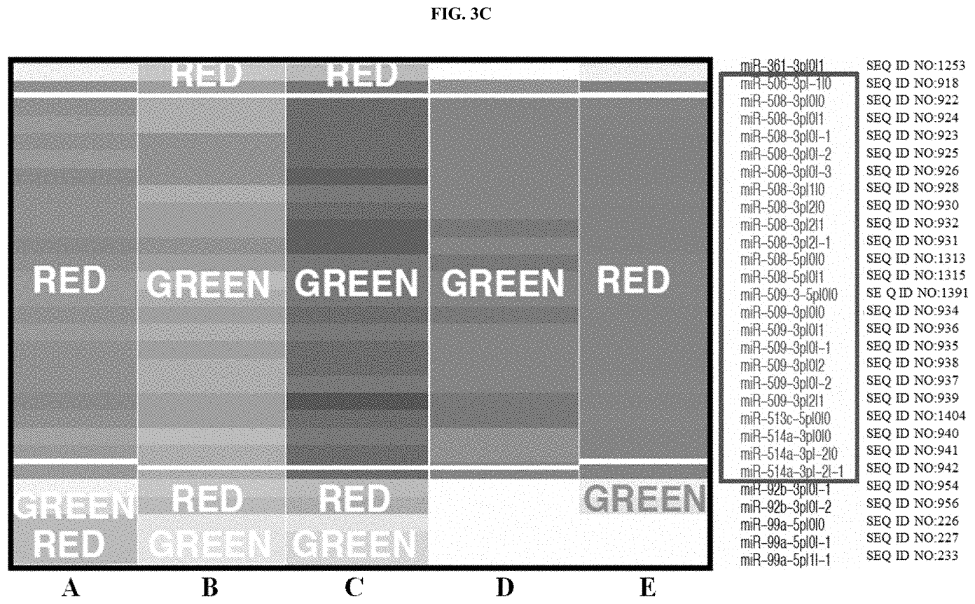

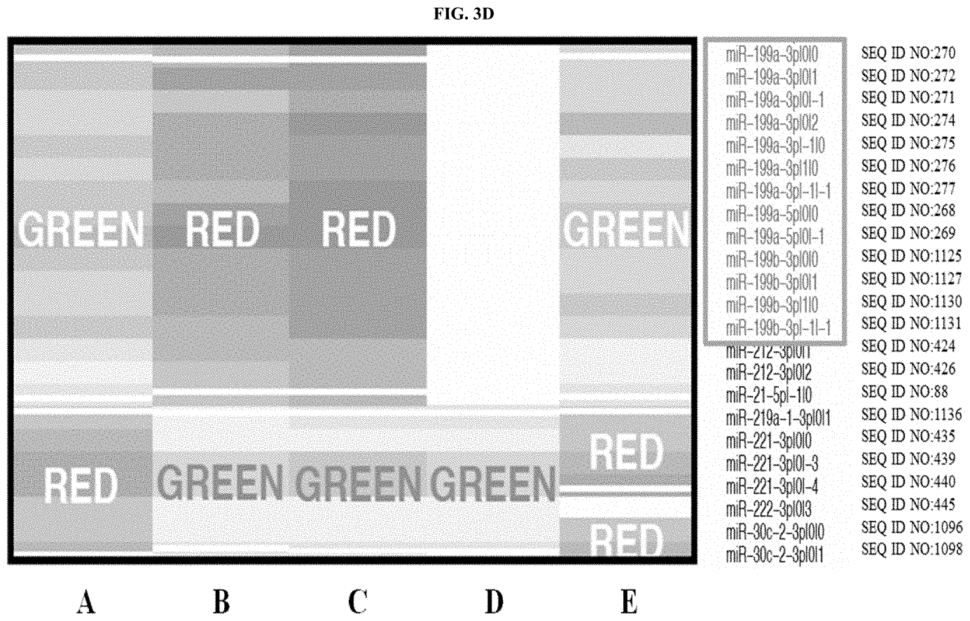

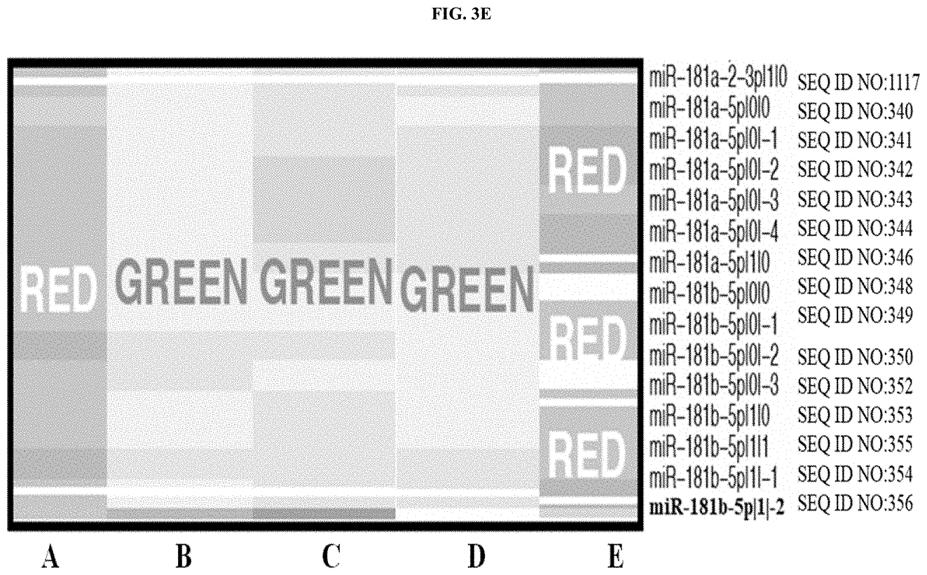

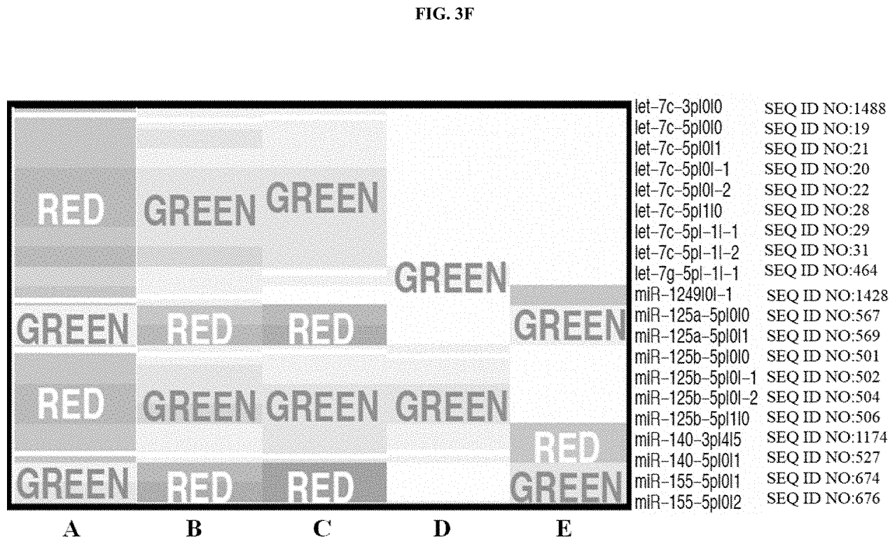

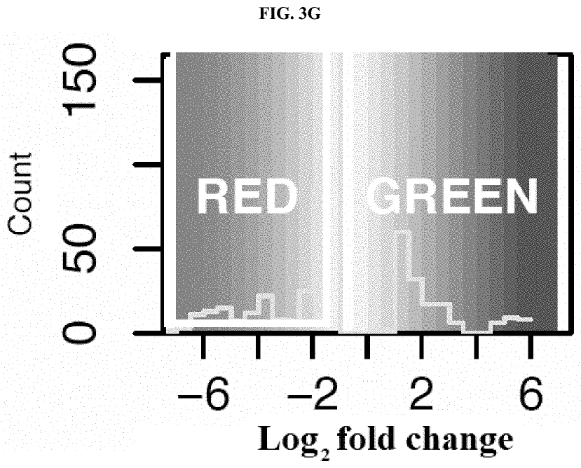

UVM datasets were stratified by M3 status and somatic BAP1 mutation status (Table 2) and searched for miRNA loci and isomiRs that are differentially abundant between the two groups. While several miRNA arms were found to be differentially abundant between these two groups, many more molecules are found to be differentially abundant when individual isomiRs are examined (FIGS. 3A-3G). Specifically, the expression of the miR-508/514 miRNA cluster from the X chromosome decreases considerably in M3 or BAP mutant patients. Additionally, the miR-508/514 cluster is not located on chromosome 3. IsomiRs from multiple other miRNA loci (e.g. miR-199a/b whose paralogues are located on chromosomes 1, 9, and 19) increase in abundance in M3 and BAP1 mutant patients (FIGS. 3A-3G). isomiRs from several de novo miRNA loci or from novel miRNA loci are also differentially abundant (FIGS. 3A-3G). This further supports the notion that these previously-unreported regulatory molecules are linked mechanistically to UVM.

TABLE 2
Summary of Clinicopathological traits

| Clinicopathological phenotype | Number of samples |
|---|---|
| Sex | Male - 45 |
| | Female - 35 |
| Chromosome 3 copy number | M3 - 37 |
| | D3 - 37 |
| Bap1 Mutation | Bap1 Mutation - 35 |
| | Bap1 WT - 45 |
| EIF1AX/SF3B1 mutations | EIF1AX/SF3B1 mutations - 27 |
| | EIF1AX/SF3B1- WT - 53 |
| Development of metastatic disease | Metastatic disease - 25 |
| | No development of metastatic |
| | disease - 54 |
| Overall prognosis | Alive - 56 |
| | Dead - 24 |
| TCGA SCNA cluster | Cluster 1 - 15 |
| | Cluster 2 - 23 |
| | Cluster 3 - 22 |
| | Cluster 4 - 20 |
| TCGA IncRNA cluster | Cluster 1 - 19 |
| | Cluster 2 - 21 |
| | Cluster 3 - 19 |
| | Cluster 4 - 21 |
| TCGA PARADIGM cluster | Cluster 1 - 14 |
| | Cluster 2 - 22 |
| | Cluster 3 - 14 |
| | Cluster 4 - 21 |
| | Cluster 5 - 5 |
| TCGA miRNA cluster | Cluster 1 - 25 |
| | Cluster 2 - 12 |
| | Cluster 3 - 27 |
| | Cluster 4 - 14 |
| | Cluster 5 - 2 |
| Histology | Epithelioid - 23 |
| | Spindle - 57 |
| AJCC tumor stage | T2a/b - 26 |
| | T3a - 27 |
| | T3b - 10 |
| | T3c/T4 - 7 |

Stratifying the UVM datasets based on protective SRSF2/SF3B1 or EIF1AX mutations reveals the opposite trend (FIGS. 3A-3G). isomiRs from the miR-508/514 miRNA cluster of chromosome X show increased abundance, whereas isomiRs from miR-199a/b and miR-142 show decreased abundance with mutation status (FIGS. 3A-3G). This is the exact opposite of the pattern observed in M3 or BAP1 mutant patients. The miR-187-3p locus offers another such example: its abundance is increased in patients with an EIF1AX or SRSF2 mutation, and decreased in patients with M3 or BAP1 mutations (FIGS. 3A-3G).

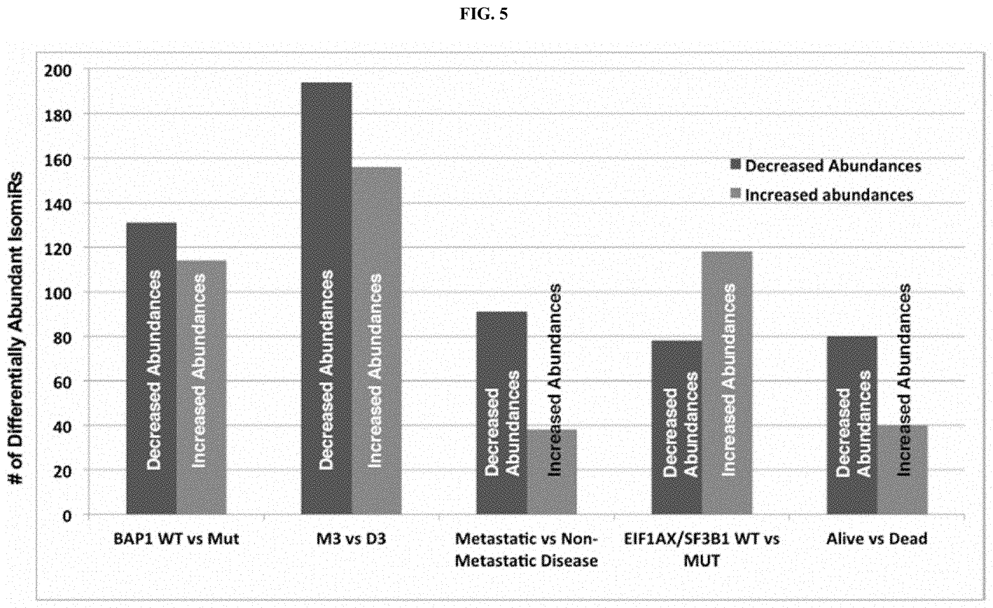

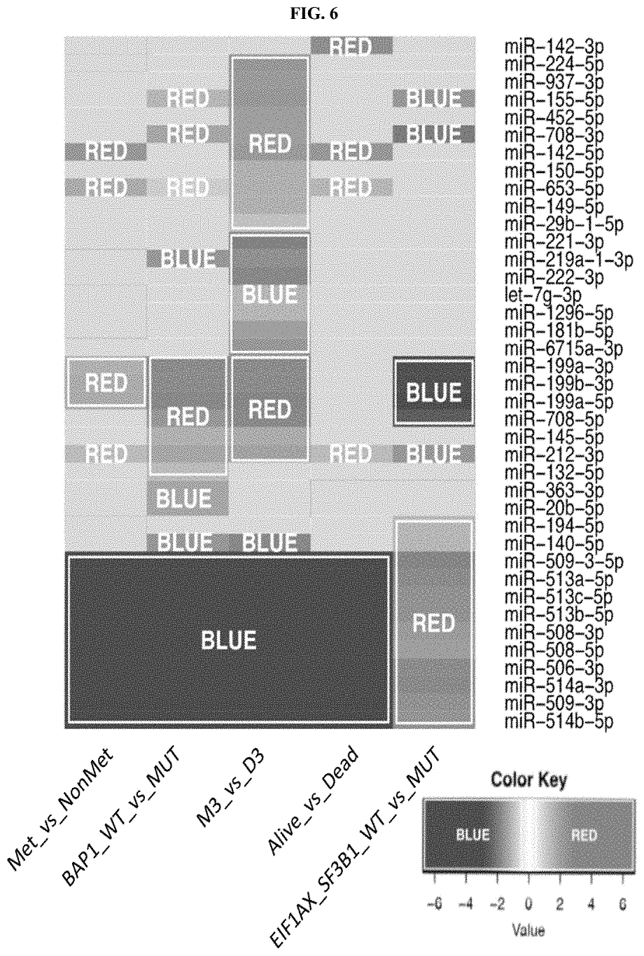

Notably, isomiRs from the same locus sometimes exhibit differing behaviors and differing associations with outcome. Consequently, it follows that the ‘functionally important’ molecules from the various loci described herein may vary among patients and will depend upon the context. FIG. 5 shows a summary of the number of isomiRs that are found to be differentially abundant in each of the comparisons. Expanding the miRNA loci yields a more complete picture of the molecules that associate to the disease phenotypes than collapsing the loci to a single miRNA arm (FIG. 6). Specifically, in many cases, isomiRs were discovered to be differentially abundant, yet their differences in abundances would therefore be missed when considering the mature locus as a whole. For example, many members of the let 7 family of miRNAs have isomiRs that are differentially abundant (FIGS. 3A-3G) whereas the locus as a whole is not differentially abundant (FIG. 6).

For completeness, the possibility that isomiRs are differentially abundant with regard to other clinical and demographic attributes was examined, including patient sex, tumor stage according to the AJCC criteria, tumor cell type as identified by histopathology, and somatic copy number analysis (SCNA), lncRNA, and PARADIGM cluster, as identified in the TCGA UVM work. While differentially abundant isomiRs were identified, they tended to be 3′ isomiRs (i.e. they shared miRNA seeds, and, thus, would be expected to have many common mRNA targets) from loci that were described by the UVM TCGA consortium.

### Example 5: Biases in tRF Lengths are Correlated with Clinical Attributes

Previous studies revealed that, for example, 18 nt tRFs can inhibit reverse transcriptase whereas 22 nt tRFs prevent their translation by blocking tRNA primer binding sites. With this in mind, it was deemed important to determine whether analogous differences exist in UVM. It was found that patients who developed metastases had a significantly higher proportion of 18-nt long tRFs (p-val=0.008) and a lower proportion of 20-nt-long tRFs (p-val=0.002) (FIG. 2D). Similarly, M3 patients showed increased proportions of 18-nt-long tRFs (p-val=0.002) (FIG. 2E). Conversely, EIF1AX mutant patients showed comparatively fewer 18-nt-long tRFs (p-val=0.021) and more 20-nt-long tRFs (p-val=0.003) (FIG. 2F). These observations suggest that tRF fragment lengths correlate with clinical features associated with patient prognosis.

### Example 6: Differential Abundance of tRFs Correlates with Clinical Attributes

Multiple tRFs were found that are associated with clinical stage, histology, development of metastatic disease, and the miRNA and lncRNA clusters reported in the TCGA UVM analyses (Table 3). Briefly, EIF1AX mutation status, sex, and development of metastasis are all binary classifications. SCNA, PARADIGM, miRNA, and lncRNA cluster designations derive from the original TCGA UVM analysis. Histology refers to the majority cell type comprising the primary tumor: either spindle, or epithelioid. Finally, AJCC staging represents primary tumor staging according to the AJCC tumor classification guidelines, with the T4 classification representing the worst prognosis.

TABLE 3
Summary of differentially abundant tRFs by clinical comparison and tRF type

| | MT | MT | MT | MT | Nuc | Nuc | Nuc | |
|---|---|---|---|---|---|---|---|---|
| | 5′-tRF | i-tRF | 3′-tRF | 5′-tRH | 5′-tRF | i-tRF | 3′-tRF | Total |
| AJCC Clinical | 0 | 0 | 0 | 0 | 1 | 3 | 0 | 4 |
| Stage | | | | | | | | |
| EIF1AX | 0 | 0 | 0 | 0 | 40 | 4 | 2 | 46 |
| Sex | 0 | 0 | 0 | 0 | 9 | 0 | 0 | 9 |
| (Male vs Female) | | | | | | | | |
| Histology | 0 | 0 | 0 | 0 | 30 | 3 | 24 | 57 |
| IncRNA_Cluster | 1 | 1 | 0 | 0 | 0 | 0 | 0 | 2 |
| Metastatic | 1 | 23 | 1 | 1 | 1 | 1 | 0 | 28 |
| Disease | | | | | | | | |
| miRNA Cluster | 1 | 33 | 2 | 0 | 3 | 0 | 0 | 39 |
| PARADIGM | 11 | 133 | 25 | 3 | 6 | 15 | 2 | 195 |
| Cluster | | | | | | | | |
| SCNA Cluster | 7 | 56 | 7 | 0 | 52 | 74 | 3 | 199 |

The PARADIGM and SCNA cluster stratifications yield the highest number of differentially abundant tRFs. Of note, the majority of these differentially abundant tRFs are i tRFs and arise from MT tRNAs. On the other hand, in SRSF2/SF3B 1 or EIF1AX mutation carriers, the majority of differentially abundant tRFs that are statistically significant are nuclear 5′-tRFs. Several differentially abundant tRFs are differentially abundant across multiple clinical and phenotypical categories (Table 4). For example, SCNA and PARADIGM clusters share several dozen differentially abundant tRFs. Interestingly, 21 tRFs are differentially abundant between “metastatic disease” and “SCNA,” or, between “metastatic disease” and “PARADIGM.” While the functional impact of these two groups of tRFs is not understood, it is reasonable to hypothesize that they may play important regulatory roles in UVM.

TABLE 4
Summary of the overlap of differentially abundant tRFs

| | EIF1AX | Sex | SCNA | IncRNA | Metastatic | Paradigm | miRNA | Histology | AJCC |
|---|---|---|---|---|---|---|---|---|---|
| EIF1AX | 46 | 1 | 27 | 0 | 0 | 2 | 0 | 2 | 0 |
| Sex | 1 | 9 | 1 | 0 | 0 | 0 | 0 | 0 | 0 |
| SCNA | 27 | 1 | 199 | 2 | 5 | 54 | 1 | 2 | 0 |
| IncRNA | 0 | 0 | 2 | 2 | 0 | 2 | 0 | 0 | 0 |
| Metastatic | 0 | 0 | 5 | 0 | 28 | 16 | 0 | 0 | 0 |
| Paradigm | 2 | 0 | 54 | 0 | 16 | 195 | 0 | 0 | 0 |
| miRNA | 0 | 0 | 1 | 0 | 0 | 1 | 39 | 0 | 0 |
| Histology | 2 | 0 | 2 | 0 | 0 | 0 | 0 | 47 | 0 |
| AJCC | 0 | 0 | 0 | 0 | 0 | 0 | 0 | 0 | 4 |

### Example 7: IsomiRs and tRFs Associate with the Development of Metastases

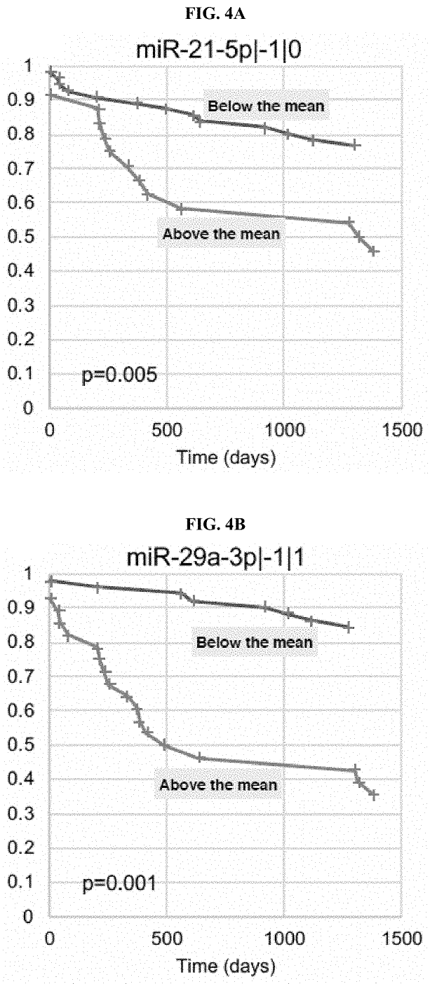

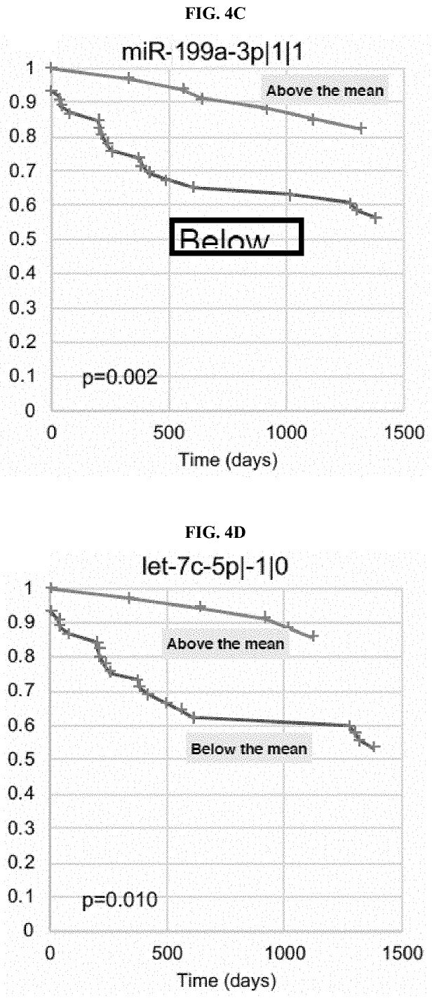

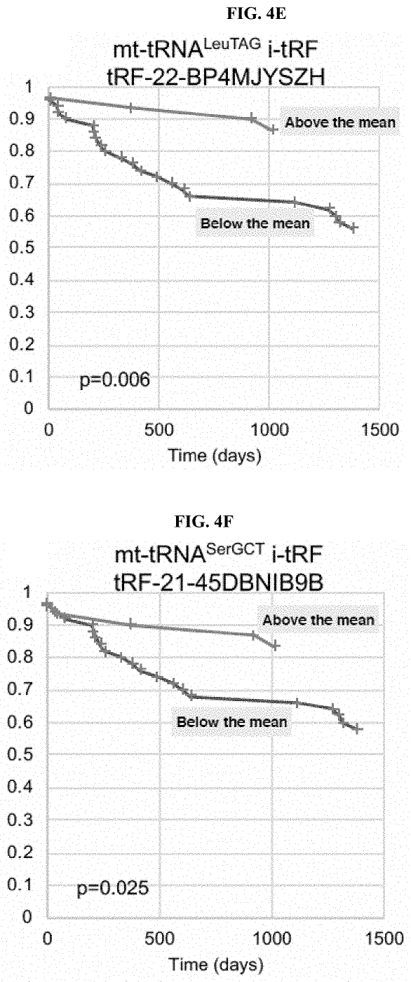

The link between individual isomiRs or tRFs and the development of metastases was examined next. Kaplan-Meier analyses were performed for time to first metastasis, grouping patients based on the mean RPM of the ncRNA in question (FIG. 4A-4D). IsomiRs such as miR-21-5p|-1|0 (SEQ ID NO:88) and miR-29a-3p|-1|1 (SEQ ID NO:161) appear increased in abundance in patients with metastases (red curves in FIG. 4A-B). In contrast, isomiRs such as miR-199a-3p|1|1 (SEQ ID NO:279) and let-7c-5p|-1|1 (SEQ ID NO:30) are decreased in abundance in patients with metastases (FIG. 4C-4D).

Lastly, tRFs that may associate with metastasis were examined. Two MT i-tRFs, tRF-22-BP4MJYSZH (SEQ ID NO:1725) from MT tRNA Leu^{TAG} and tRF-21-45DBNIB9B (SEQ ID NO:2921) from MT tRNA Ser^{GCT}, showed significant differences in time to first metastasis when patients were stratified based on the tRFs' respective mean RPM values (FIG. 4E-4F). Naturally, these tRFs and isomiRs are interesting targets for future experimental investigations.

The recent TCGA analysis of UVM identified four main clinical subcategories associated with patient outcomes. The two categories with poorer prognosis were characterized by M3 status and BAP1 mutations. Patients with these characteristics show increased overall risk of progressing to metastatic disease. Patients with D3 and EIF1AX/SF3B1 mutations have decreased metastatic risk and better disease prognosis. To gain additional insights into the molecular architecture of this disease, TCGA datasets were re-analyzed, specifically focusing on characterizing the profiles and expression of isomiRs and tRFs. These two categories of short ncRNAs have received considerable attention recently, because of their increasingly evident involvement in key regulatory processes.

The study of the isomiR and tRF profiles described herein reveals a complex regulatory network active in UVM. In several instances, it was found that their expression correlates with distinct clinical outcomes. This exploration of isomiRs and tRFs in this disease has identified many novel features that can be further explored as biomarkers for diagnostic and therapeutic purposes.

Previous miRNA profiling endeavors in UVM characterized the expression of numerous highly abundant miRNA loci. In addition to corroborating these earlier observations, this work goes significantly further than previous efforts in identifying novel and important features of the UVM miRNA-ome. For example, it has been shown that 72.5% of the 407 miRNA loci that are active in UVM express multiple isomiRs (Table 1, above). In fact, for nearly half of these loci, the most abundant isoform is not the archetype isoform that is listed in miRBase. Moreover, for 21.3% of these loci the archetype isoform listed in miRBase is not even present in the UVM samples. As a result, many of the isoforms that are important for UVM biology have not been studied before, while many of the isoforms that have been studied to date were either not the most relevant ones or absent altogether. These two observations strongly suggest that a very large part of the regulatory layer of UVM (isomiRs and their mRNA targets) has not been studied.

Adding to this last point, the 5′ termini of ˜30% of all isomiRs that emerged from the present analysis of UVM differ from the 5′ termini of the respective archetypal miRNAs that have been studied to date. Because a shifted 5′ terminus creates a change in the miRNA's seed sequence, the discovery of these isomiRs is of functional consequence. These isomiRs have mRNA targetomes whose contribution to the UVM biology is uncharacterized and differs from what has been studied to date. Indeed, as previously demonstrated in the context of breast cancer, different isomiRs from the same miRNA arm have largely non-overlapping mRNA targetomes.

The 3p arm of miR-140 (FIG. 1B) offers a characteristic example of the relevance of these studies. Of the 18 isomiRs from this locus that are active in UVM, four are at least as abundant as the archetype and their 5′ terminus differs from that of the archetype. Previous work showed that a 5′-isomiR from miR-140-3p is increased in breast tumors compared to normal breast tissue, wherein it functions to suppress tumor growth and progression. The four highly abundant 5′-isomiRs identified herein share the same seed as the isomiR that was examined in the breast cancer study. Interestingly, these four isomiRs are increased in abundance in patients with EIFA1X or SF3B1 mutations, and decreased in M3 and BAP1 mutation carriers. This suggests that these isomiRs may play tumor-suppressive roles in UVM, just as in breast cancer, an observation that remains to be validated.

Previous reports provided thousands of novel miRNA loci that are both tissue- and primate-specific. It has been found that many of these previously-reported miRNA loci produce abundant isomiRs in UVM and that their expression correlates with clinical attributes (FIGS. 3A-3G). De novo analyses of the UVM samples uncovered 32 novel miRNA loci. Several of these loci appear to be expressed in a tissue-specific manner. While the functional importance of these newly discovered miRNAs remains to be determined, they nonetheless have potential application as disease-specific biomarkers.

As shown in FIGS. 3A-3G, multiple loci and isomiRs are differentially abundant across clinical attributes. The miR-508/514 locus stands out. Several of the 15 miRNAs in this cluster have been associated with functions upstream of several pathways involved in tumor development, promotion of melanocyte transformation, and melanoma growth. Furthermore, over-expression of some of these miRNAs in skin melanoma (SKCM) results in decreased cell proliferation and colony formation. Individual miRNAs of this cluster have been associated with cellular phenotypes in other cancers. For example, miR-509-3p has been shown to inhibit cellular migration in ovarian cancer cells. Decreased expression of this miRNA, as seen in UVM, may increase metastatic potential. Similarly, low abundances of miR-508-5p in glioma have been associated with shorter overall survival. Inhibition of miR-508-5p results in increased cell proliferation and cellular migration. Interestingly, this cluster shows the opposite effect in patients with protective SIF1AX and SF3B1 mutations. Therefore, decreased expression here may be indicative of poor prognosis, perhaps arising from promotion of tumor growth. It should be noted that only ˜5% of all of the differentially abundant isomiRs reside on chromosome 3. Since M3 status correlates with poorer overall survival, it is expected that many of the miRNA loci residing on this chromosome would be differentially abundant. At the same time, the large number of differentially abundant isomiRs that are derived from other chromosomes suggests that M3 status makes a relatively small contribution to the spectrum of differentially abundant isomiRs.

Finally, described herein is the finding that multiple isomiRs are associated with patient survival (FIG. 4A-D). Combined, these collections of isomiRs have a potential application as disease biomarkers that may help determine patient outcomes and/or to decide when to begin new therapeutic interventions.

It is also worth mentioning here an interesting interplay between the abundances of individual isomiRs from a miRNA arm and the abundance of the arm as a whole. Specifically, multiple instances of isomiRs that are differentially abundant between two groups of patients, but whose corresponding miRNA arm as a whole did not show any change in abundance have been found. This finding highlights the importance of studying miRNA regulation at the isomiR level and its ability to uncover new consequential players, in UVM biology and elsewhere. Doing so is likely to reveal novel disease dependencies that would have otherwise remained hidden from view.

In addition to exploring the UVM miRNA-ome, the endogenous tRFs have been profiled and analyzed, the results of which are in line with earlier findings that tRF dysregulation is present in other disease types. The analyses described herein show that numerous tRFs are expressed at high levels in UVM. tRFs of nuclear origin are mostly 5′-tRFs, whereas MT tRFs are predominantly 3′-tRFs (FIG. 2A). Additionally, nearly three quarters of the UVM tRFs are produced by ten tRNA isoacceptors: MT ValTAC, MT TyrGTA, Nuc HisGTG, Nuc GluTTC, Nuc ValCAC, Nuc GlyGCC, MT ProTGG, Nuc GluCTC, Nuc AlaCGC, and Nuc GlnCTG (FIG. 2B). Considering the tRF studies in other contexts, this particular tRNA isoacceptor bias is unique to UVM. It is conceivable that the corresponding tRFs are particularly important for key regulatory pathways in UVM and further work is required to determine the full functional consequences of this observation.

The present analysis also revealed a length bias in the production of tRFs (FIG. 2). MT-derived tRFs exhibit a unimodal distribution with a peak at 20 nts (FIG. 2C). Nuclear tRFs on the other hand exhibit a bimodal distribution with peaks at 20 and 23 nts (FIG. 2C). This length bias suggests that distinct processes produce tRFs from the MT and nuclear tRNAs. Recall here that the sequencing protocol employed by the TCGA likely prevents observation of tRFs longer than 30nt.

Patients who developed metastases have a significantly higher percentage of tRFs of length 18 nts and a significantly lower percentage of tRFs of length 20 nts (FIGS. 2C-2D). Similarly, M3 patients show an increased percentage of 18-nt-long tRFs. Conversely, patients with protective EIF1AX mutations show a decrease in 18-nt-long tRFs and an increase in 20-nt-long tRFs. The mechanisms responsible for these cleavage patterns are not known. Previous work described a mechanism whereby 18-nt-long fragments block reverse transcription whereas longer tRFs (22 nts) prime RNAi mediated degradation. Thus, the strong association between tRF length and clinical characteristics warrants determination of the functional roles of these tRFs in UVM.

Lastly, a large number of differentially abundant tRFs were identified across clinical categories (Table 3). tRF profiles differ across the formerly-established somatic copy number analysis (SCNA) and mRNA paradigm cluster (PARADIGM) analysis described by the TCGA Consortium. Additionally, tRFs were found to be differentially abundant in the context of EIF1AX mutations, metastatic disease, and when considering the majority cell type of the underlying tumor. The vast majority of tRFs are differentially abundant in one category, but a number are shared across categories. tRF abundance also associates with overall patient survival (FIG. 4E-4F). This evidence points toward differential activity of tRFs in each of these contexts. Unique tRFs may play a role in regulation of pro-metastatic pathways, whereas others may be active in the control and differentiation of uveal cells.

Taken together, these findings delineate complex relationships among the isomiRs and tRFs that are present in primary UVM. The present disclosure provides many novel features that can be potentially leveraged to build novel diagnostic, prognostic, or therapeutic applications. While much additional work is needed to elucidate the roles of these molecules, the generated insights represent a first step towards making new inroads into improving patient outcomes in UVM.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a method for assessing whether a uveal melanoma (UVM) patient has, or is at risk of developing a condition, the method comprising:
(a) measuring in a biological sample obtained from the patient the abundance of at least one of:
   (i) at least two isoforms (isomiRs) of at least one miRNA; and
   (ii) at least one tRNA derived fragment (tRF);
(b) computing a difference in the abundance of at least one of:
   (i) the at least two isomiRs of at least one miRNA; and
   (ii) the at least one tRF;
in the biological sample as compared to the abundance of the same molecules in a reference sample,
   wherein the difference that results from the computing is an indication that the subject either has, or is at risk of developing, or is at a given stage of the condition.

Embodiment 2 provides the method of Embodiment 1, wherein the condition is metastasis.

Embodiment 3 provides the method of any of Embodiments 1-2, wherein each isomiR of the at least two isomiRs is encoded by a sequence selected from the group consisting of SEQ ID NOs:1-1677.

Embodiment 4 provides the method of any of Embodiments 1-3, wherein each tRF of the at least one tRF is encoded by a sequence selected from the group consisting of SEQ ID NOs:1678-5511.

Embodiment 5 provides the method of any of Embodiments 1-4, wherein the abundance of both the at least two isomiRs and the at least one tRF are measured in the biological sample.

Embodiment 6 provides the method of any of Embodiments 1-5, wherein if the abundance of the at least two isomiRs in the biological sample differ from the abundance of the same molecules in the reference sample, the patient is determined as having, being at risk of developing, or being at a given stage of the condition.

Embodiment 7 provides the method of any of Embodiments 1-6, wherein the abundance of the at least two isomiRs in the biological sample are greater than the abundance of the same molecules in the reference sample.

Embodiment 8 provides the method of any of Embodiments 1-7, wherein at least one of the at least two isomiRs is encoded by a sequence selected from the group consisting of SEQ ID NO:88 and SEQ ID NO:161.

Embodiment 9 provides the method of any of Embodiments 1-8, wherein the abundance of the at least two isomiRs in the biological sample are less than the abundance of the same molecules in the reference sample.

Embodiment 10 provides the method of any of Embodiments 1-9, wherein at least one of the at least two isomiRs is encoded by a sequence selected from the group consisting of SEQ ID NO:279 and SEQ ID NO:30.

Embodiment 11 provides the method of any of Embodiments 1-10, wherein if the abundance of the at least one tRF in the biological sample differ from the abundance of the same molecules in the reference sample, the patient is determined as having, being at risk of developing, or being at a given stage of the condition.

Embodiment 12 provides the method of any of Embodiments 1-11, wherein if the abundance of both of the at least two isomiRs and the at least one tRF in the biological sample differ from the abundance of the same molecules in the reference sample, the patient is determined as having, being at risk of developing, or being at a given stage of the condition.

Embodiment 13 provides the method of any of Embodiments 1-12, wherein the difference comprises a ratio of the abundance of the at least two isomiRs in the biological sample and the abundance of the same molecules in the reference sample, wherein the ratio has a log 2 value with an absolute value greater than or equal to 1.

Embodiment 14 provides the method of any of Embodiments 1-13, wherein the difference comprises a ratio of the abundance of the at least one tRF in the biological sample and the abundance of the same molecules in the reference sample, wherein the ratio has a log 2 value with an absolute value greater than or equal to 1.

Embodiment 15 provides the method of any of Embodiments 1-14, wherein the measuring comprises subjecting the biological sample to deep sequencing.

Embodiment 16 provides the method of any of Embodiments 1-15, wherein the measuring comprises subjecting the biological sample to a polymerase chain reaction (PCR).

Embodiment 17 provides the method of any of Embodiments 1-16, wherein the PCR can ensure the identity of the endpoints of the molecule being measured.

Embodiment 18 provides the method of any of Embodiments 1-17, wherein the PCR comprises a modified quantitative reverse transcription PCR (qRT-PCR).

Embodiment 19 provides the method of any of Embodiments 1-18, further comprising recommending a therapeutic regimen.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A method for assessing whether a uveal melanoma (UVM) patient has, or is at risk of developing a condition, the method comprising:
(a) measuring in a biological sample obtained from the patient the abundance of at least one of:
(i) at least two isoforms (isomiRs) of at least one miRNA; and
(ii) at least one tRNA derived fragment (tRF);
(b) computing a difference in the abundance of at least one of:
(i) the at least two isomiRs of at least one miRNA; and
(ii) the at least one tRF;
in the biological sample as compared to the abundance of the same molecules in a reference sample,
wherein the difference that results from the computing is an indication that the subject either has, or is at risk of developing, or is at a given stage of the condition.

2. The method of claim 1, wherein the condition is metastasis.

3. The method of claim 1, wherein each isomiR of the at least two isomiRs is encoded by a sequence selected from the group consisting of SEQ ID NOs:1-1677.

4. The method of claim 1, wherein each tRF of the at least one tRF is encoded by a sequence selected from the group consisting of SEQ ID NOs:1678-5511.

5. The method of claim 1, wherein the abundance of both the at least two isomiRs and the at least one tRF are measured in the biological sample.

6. The method of claim 1, wherein if the abundance of the at least two isomiRs in the biological sample differ from the abundance of the same molecules in the reference sample, the patient is determined as having, being at risk of developing, or being at a given stage of the condition.

7. The method of claim 6, wherein the abundance of the at least two isomiRs in the biological sample are greater than the abundance of the same molecules in the reference sample.

8. The method of claim 7, wherein at least one of the at least two isomiRs is encoded by a sequence selected from the group consisting of SEQ ID NO:88 and SEQ ID NO:161.

9. The method of claim 6, wherein the abundance of the at least two isomiRs in the biological sample are less than the abundance of the same molecules in the reference sample.

10. The method of claim 9, wherein at least one of the at least two isomiRs is encoded by a sequence selected from the group consisting of SEQ ID NO:279 and SEQ ID NO:30.

11. The method of claim 1, wherein if the abundance of the at least one tRF in the biological sample differ from the abundance of the same molecules in the reference sample, the patient is determined as having, being at risk of developing, or being at a given stage of the condition.

12. The method of claim 1, wherein if the abundance of both of the at least two isomiRs and the at least one tRF in the biological sample differ from the abundance of the same molecules in the reference sample, the patient is determined as having, being at risk of developing, or being at a given stage of the condition.

13. The method of claim 1, wherein the difference comprises a ratio of the abundance of the at least two isomiRs in the biological sample and the abundance of the same molecules in the reference sample, wherein the ratio has a log 2 value with an absolute value greater than or equal to 1.

14. The method of claim 1, wherein the difference comprises a ratio of the abundance of the at least one tRF in the biological sample and the abundance of the same molecules in the reference sample, wherein the ratio has a log 2 value with an absolute value greater than or equal to 1.

15. The method of claim 1, wherein the measuring comprises subjecting the biological sample to deep sequencing.

16. The method of claim 1, wherein the measuring comprises subjecting the biological sample to a polymerase chain reaction (PCR).

17. The method of claim 16, wherein the PCR can ensure the identity of the endpoints of the molecule being measured.

18. The method of claim 16, wherein the PCR comprises a modified quantitative reverse transcription PCR (qRT-PCR).

19. The method of claim 1, further comprising recommending a therapeutic regimen.
